(19) United States
(12) Patent Application Publication
(10) Pub. No.: US 2022/0047455 A1
(43) Pub. Date: Feb. 17, 2022

(54) EMERGENCY CARDIAC ARREST RESUSCITATION DRONE SYSTEM

(71) Applicant: Brenton Strauch, Des Moines, IA (US)

(72) Inventor: Brenton Strauch, Des Moines, IA (US)

(21) Appl. No.: 17/399,701

(22) Filed: Aug. 11, 2021

Related U.S. Application Data
(60) Provisional application No. 63/064,617, filed on Aug. 12, 2020.

(51) Int. Cl. A61H 31/00; G16H 40/40; G16H 40/67; B64C 39/02

(52) U.S. Cl. CPC A61H 31/006; A61H 31/005; G16H 40/40; G16H 40/67; B64C 2201/208; A61H 2230/04; B64C 2201/127; B64C 2201/145; B64C 39/024

(57) ABSTRACT

An emergency cardiac arrest resuscitation drone system that includes a drone, a mechanical chest compression device that is attached to the drone, and a control system connected to the drone. The control system is configured to activate and direct the drone to a geographic location of an emergency. The drone has a GPS system, a microphone/speaker, and a camera that enable a medical certified staff member to provide step by step instructions to a bystander at the geographic location of the emergency.

### CROSS REFERENCE TO RELATED APPLICATION

This application claims the benefit of U.S. Provisional Application Ser. No. 63/064,617 filed Aug. 12, 2020, the contents of this application is hereby incorporated by reference in their entirety.

### BACKGROUND OF THE INVENTION

This invention is directed to an emergency cardiac arrest resuscitation drone system and more particularly, a system that provides people that go into cardiac arrest in out-of-hospital settings so bystanders, in a fraction of the time it would take first responders to arrive, can provide proper chest compressions giving the patient a higher chance of surviving cardiac arrest.

In busy metropolitan areas, where traffic is heavy, it can take first responders up to 20 minutes to arrive at the scene of a cardiac arrest emergency. In rural areas the closest hospital may be an hour away and some small towns do not have a local EMS. In emergency situations, this leaves a family member, friend, or bystander limited options. The average bystander is not prepared to perform CPR and if they do try, it is unlikely that they perform the process correctly. CPR is much easier now that mouth-to-mouth resuscitation is no longer recommended. While it's true that many people don't survive cardiac arrest—the numbers vary study to study, but some find rates of survival for out-of-hospital cardiac arrest at less than 5%. The chances for survival are higher with bystander help. The AHA, in December of 2014, stated that the survival rate of out of hospital cardiac arrest is 10.6%. Instructions regarding the appropriate use of CPR and automated external defibrillators (AEDs) are not complicated and are widely available. Further, mechanical chest compression devices are known in the art and make performing cardiac arrest procedures easier and more uniform.

The problem is getting the mechanical chest compression device to a remote site in time to do good. Delivering the device within minutes can greatly reduce mortality rates in pre-hospital settings, even before emergency services can arrive. Instead of waiting 15 to 30 minutes for emergency crews to arrive, it is desired to provide the equipment more quickly, ideally within five minutes. Accordingly, a need exists in the art for system that will address the deficiencies.

Therefore, an objective of the present invention is to provide an emergency cardiac arrest resuscitation drone system to deliver a mechanical chest compression device to an emergency location more quickly.

Another objective of the present invention is to provide an emergency cardiac arrest resuscitation drone system configured to permit a medical certified staff member to provide step by step instructions to a bystander at an emergency location.

A still further objective of the present invention is to provide an emergency cardiac arrest resuscitation drone system configured to transmit real time feedback to a remote location.

These and other objectives will be apparent to those having ordinary skill in the art based upon the following written description, drawings, and claims.

### SUMMARY OF THE INVENTION

An emergency cardiac arrest resuscitation drone system includes a drone, a mechanical chest compression device attached to the drone, and a control system connected to the drone. The control system is configured to activate and direct the drone to a geographic location of an emergency. The drone has a GPS system, a microphone/speaker, and a camera that enable a medical certified staff member to provide step by step instructions to a bystander at the geographic location of the emergency.

The mechanical chest compression device includes EKG sensors and is configured to transmit real time feedback to a remote location. The drone is activated by the control system based upon GPS coordinates and an emergency call transmitted from a dispatch. Further, the drone is stationed on top of a building, on a tower placed in a field, or on a mobile launch platform on top of a vehicle.

### BRIEF DESCRIPTION OF THE DRAWINGS

FIG. 1 is a schematic view of an emergency cardiac arrest resuscitation drone system; and

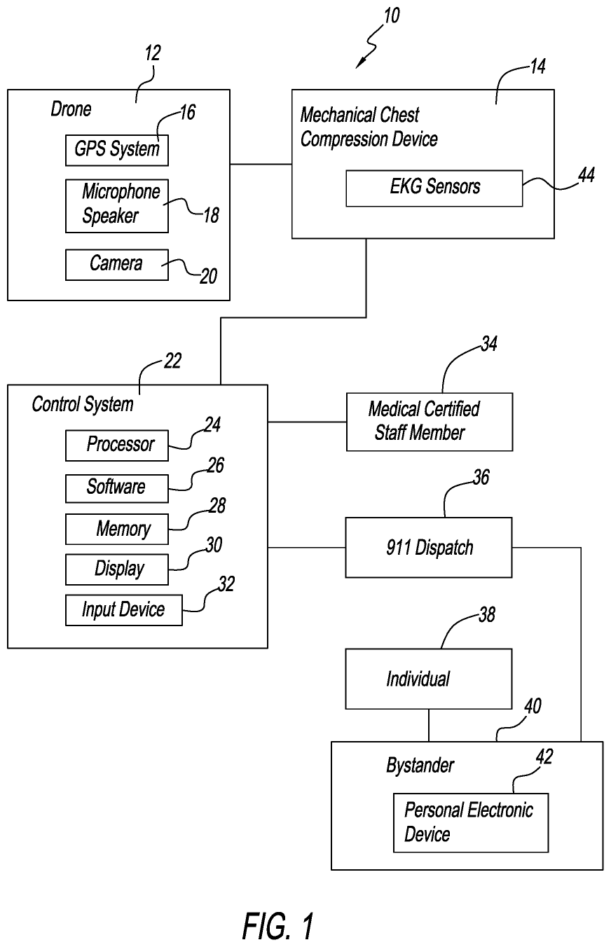

FIG. 2 is a flow diagram of an emergency cardiac arrest resuscitation drone system.

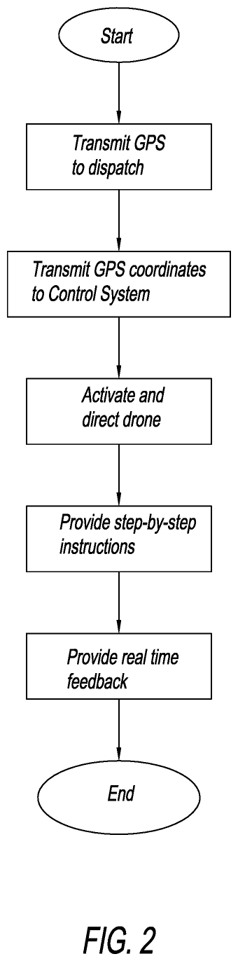

### DETAILED DESCRIPTION

With reference to the figures an emergency cardiac arrest resuscitation drone system 10 includes a drone 12 having auto pilot capabilities. Attached to the drone 12 is a mechanical chest compression device 14. The mechanical chest compression device is of any type and preferred is LUCAS® device which is an easy-to-use mechanical chest compression device that helps lifesaving teams around the world deliver high-quality, guidelines-consistent chest compression to sudden cardiac arrest patients; in the field, on the move and in the hospital. The LUCAS® device has been shown to improve the quality of chest compressions, increase ETCOS levels, as well as being able to sustain life-saving circulation during prolonged resuscitation attempts. The LUCAS® device has been studied extensively and been shown to be safe, effective, and able to save those patients that would have otherwise been considered futile. Alternatively, the Zoll Auto Pulse™ can be used.

The drone 12 also has a GPS system 16, a microphone/speaker 18, and a camera 20. The drone 12 and its components are connected to a control system 22 that includes a processor 24, software 26, memory 28, a display 30 and an input device 32. The control system 22, operated by a medically certified staff member 34, is connected to the drone 12, the GPS system 16, the microphone/speaker 18, the camera 20, and 911 dispatch 36 or the like.

In operation, when an individual 38 is in cardiac arrest a bystander 40, using a personal electronic device 42 such as a mobile phone or the like contacts 911 dispatch 36 to report an emergency. The mobile phone 42 transmits GPS coordinates to the 911 dispatch 36. The emergency call and GPS coordinates are transmitted to the control system 22 wherein the drone 12 is activated. Using auto pilot capabilities, the drone 12 flies to the geographic location of the emergency and lands. Once the drone arrives the medical certified staff member 34 is able to speak through the microphone/speaker 18 and see through the camera 20 the bystander 40 and the individual 38. The medical certified staff member 34 is able to provide step by step instructions to the bystander 40 on how to apply the mechanical chest compression device 14 to the individual 38. As a result, bystanders 40 are less worried about doing the wrong thing and causing further damage.

EKG sensors 44 will be placed in a back panel and side panels of the mechanical chest compression device 14 to automatically detect if the individual 38 has a viable heart rhythm while also allowing the device 14 to automatically stop and pause. Real time feedback is transmitted to 911 dispatch 36 and the medical certified staff member 34, preferably through Wi-Fi and Bluetooth. This information is also provided to ER physicians and EMS professionals to permit pre-monitoring in route, provide an opportunity to have a plan lined up for what to do next, and permit direct advice to medical crews before arriving at a hospital. Additional equipment can be attached to the drone 12 such as Stop the Bleed Kits, Birthing Kits, general first aid kits, and the like.

The drones 12 are stationed on top of buildings, on mobile launch platforms on top of ambulances or fire engines, or towers placed in fields in order to cut down on response time. The drones would be able to dock and charge and be ready to go and a moment's notice.

From the above discussion and accompanying figures and claims it will be appreciated that the emergency cardiac arrest resuscitation drone system 10 offers many advantages over the prior art. It will be appreciated further by those skilled in the art that other various modifications could be made to the device without parting from the spirit and scope of this invention. All such modifications and changes fall within the scope of the claims and are intended to be covered thereby. It should be understood that the examples and embodiments described herein are for illustrative purposes only and that various modifications or changes in the light thereof will be suggested to persons skilled in the art and are to be included in the spirit and purview of this application.

What is claimed is:

1. An emergency cardiac arrest resuscitation drone system, comprising:
a drone;
a mechanical chest compression device attached to the drone;
a control system connected to the drone;
wherein the control system activates and directs the drone to a geographic location of an emergency.

2. The system of claim 1 wherein the drone has a GPS system, a microphone/speaker, and a camera that enable a medical certified staff member to provide step by step instructions to a bystander at the geographic location of the emergency.

3. The system of claim 1 wherein the mechanical chest compression device includes EKG sensors.

4. The system of claim 1 wherein the mechanical chest compression device transmits real time feedback.

5. The system of claim 1 wherein the drone is activated by the control system based upon GPS coordinates and an emergency call transmitted from a dispatch.

6. The system of claim 1 wherein the drone is stationed on a mobile launch platform on top of a vehicle.

7. The system of claim 1 wherein the drone is stationed on top of a building.

8. The system of claim 1 wherein the drone is stationed on a tower placed in a field.

9. The system of claim 1 wherein the drone is configured to dock and charge.
